(19) United States
(12) Patent Application Publication
(10) Pub. No.: US 2025/0009206 A1
(43) Pub. Date: Jan. 9, 2025

(54) TWO-WAY FLUIDICS VALVE FOR BRONCHOSCOPY

(71) Applicant: Boston Scientific Scimed, Inc., Maple Grove, MN (US)

(72) Inventors: Daniel Joseph Faulkner, Framingham, MA (US); Richard Paul Mansfield, Sterling, MA (US)

(73) Assignee: Boston Scientific Scimed, Inc., Maple Grove, MN (US)

(21) Appl. No.: 18/760,476

(22) Filed: Jul. 1, 2024

Related U.S. Application Data
(60) Provisional application No. 63/511,772, filed on Jul. 3, 2023.

(51) Int. Cl. A61B 1/00; A61B 1/015; A61B 1/267

(52) U.S. Cl. CPC A61B 1/00068; A61B 1/015; A61B 1/2676

(57) ABSTRACT

Devices, systems, and methods for a two-way valve assembly for a medical device. The two-way valve assembly has a knob seated within a valve body, two inlet passages leading to a suction source and a gas source, an outlet passage leading to an endoscope fluid channel, and an internal channel connecting one of the two inlet passages to the outlet passage. A spring presses the knob into the valve body to seal the openings along the channels and passages. Rotating the knob slides one or more guide pins on the knob along one or more guide grooves on the body and connects the other inlet passage to the outlet passage, such that the same endoscope fluid channel can be used both for suction, as in clearing fluid from within a patient, and delivering gas, as in insufflation.

### CROSS REFERENCE TO RELATED APPLICATIONS

This application claims the benefit of U.S. Provisional Patent Application Ser. No. 63/511,772 filed on Jul. 3, 2023, the disclosure of which is incorporated herein by reference.

### TECHNICAL FIELD

The present disclosure generally relates to endoscope suction tubing. Particularly, but not exclusively, the present disclosure relates to a valve for two-way fluidics within an endoscope.

### BACKGROUND

Endoscopic examinations provide a minimally-invasive procedure for visual investigation of internal bodily lumens. Bronchoscopy is an endoscopic diagnostic technique for directly examining the airways of the lungs via insertion of a long, thin endoscope (or bronchoscope) through the trachea of a patient and down into the lung pathways. A feature present in some bronchoscopes is a suction tube that allows for evacuation of fluid from the airways before and during examination.

Another procedure that often precedes or succeeds bronchoscopy is insufflation, in which gas (often oxygen, air, or carbon dioxide) is supplied at a positive pressure to the airways. Examination of insufflated airways can allow for detection of air leaks in the patient's respiratory system.

Switching between endoscopy and insufflation may require unnecessary time and cause extra trauma. Thus, there is a need for a device that allows for a medical professional to apply both suction and insufflation as needed during a bronchoscopy procedure.

### SUMMARY

This disclosure provides design, material, manufacturing method, and use alternatives for medical devices and medical systems. In a first example, two-way valve assembly for a medical device can comprise a knob, a valve body, and a spring. The knob includes a knob barrel having a side wall and an internal channel running between inlet and outlet openings in the side wall and one or more guide pins. The valve body includes a valve well encircling the knob barrel, first and second inlet passages in fluid communication with the valve well, an outlet passage in fluid communication with the valve well, and one or more guide grooves, each guide groove receiving a different one of the one or more guide pins. The spring applies force to press the side wall of the knob barrel against an inner surface of the valve well. The knob rotates relative to the valve body between a first position in which the inlet opening in the knob barrel forms a seal with the first inlet passage in the valve body and the outlet opening in the knob barrel forms a seal with the outlet passage in the valve body, placing the first inlet passage in fluid communication with the outlet passage through the internal channel of the knob, and a second position in which the inlet opening in the knob barrel forms a seal with the second inlet passage in the valve body and the outlet opening in the knob barrel forms a seal with the outlet passage in the valve body, placing the second inlet passage in fluid communication with the outlet passage through the internal channel of the knob. Each of the one or more guide pins moves from a first seat in a guide groove corresponding to the first position of the knob to a second seat of the guide groove corresponding to the second position of the knob when the knob rotates.

Alternatively or additionally to any of the examples above, the valve assembly can further include an end cap attached to the valve body opposite the knob, wherein the spring is attached to the valve body and the end cap to push the end cap away from the valve body, pressing the knob into the valve body.

Alternatively or additionally to any of the examples above, the knob can further include a pole extending through the valve body and into the end cap, the pole having a key fitting at its distal end shaped to engage with a keyhole in the end cap.

Alternatively or additionally to any of the examples above, the valve assembly can further include a tube within the internal channel of the knob barrel, the tube having raised portions at the inlet and outlet openings to form a seal when the knob barrel is pressed against the valve well.

Alternatively or additionally to any of the examples above, the outlet opening of the knob barrel can subtend an angle of the side wall equal to or greater than the angle of rotation between the first position and the second position.

Alternatively or additionally to any of the examples above, the guide grooves can each be shaped with a raised portion between the first seat corresponding to the first position of the knob and the second seat corresponding to the second position of the knob such that, when rotating the knob between the first position and the second position, the knob barrel is lifted relative to the valve well to break contact between the knob barrel and the valve well.

Alternatively or additionally to any of the examples above, the one or more guide pins can a plurality of guide pins extending radially from the knob and spaced with radial symmetry about the knob, and the one or more guide grooves can be a plurality of guide grooves shaped and spaced with radial symmetry about the valve body.

Alternatively or additionally to any of the examples above the side wall of the knob barrel and the inner wall of the valve body can be cylinders tapered from top to bottom.

Alternatively or additionally to any of the examples above, the knob can include a handle sized and shaped to be grasped and rotated by one hand.

In another example, a medical system for bronchoscopy comprises an endoscope having a fluid channel therein, a two-way valve assembly according to any of the examples above in fluid communication with the endoscope fluid channel, a source of suction in fluid communication with the first inlet passage of the two-way valve assembly such that, when the two-way valve assembly is in a first position, suction is applied through the endoscope fluid channel; and a gas source in fluid communication with the second inlet passage of the two-way valve assembly such that, when the two-way valve assembly is in a second position, gas is supplied through the endoscope fluid channel.

Alternatively or additionally to any of the examples above, the endoscope can be a bronchoscope suitable for diagnostic and/or surgical deployment in the human respiratory system.

Alternatively or additionally to any of the examples above the gas source can be source of gas suitable for insufflation during bronchoscopy.

Alternatively or additionally to any of the examples above, the gas can be room air, contained air, or oxygen.

In another example, a method for operating a medical system can use any of the medical systems described above and comprises the steps of: deploying the endoscope into the respiratory system of a patient; while the two-way valve is in a first position, clearing fluid from the respiratory system of the patient via suction through the endoscope fluid channel; rotating the knob of the two-way valve assembly from the first position to the second position; and insufflating the patient with gas deployed through the endoscope fluid channel.

Alternatively or additionally to any of the examples above, rotating the knob of the two-way valve assembly from the first position to the second position can be done with one of a user's hands while the user's other hand is operating the endoscope.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a partial perspective view of an illustrative endoscope handle and two-way valve;

FIG. 3 depicts an exploded perspective view of an illustrative two-way valve;

FIG. 4 depicts a perspective view of an illustrative valve knob;

FIG. 5 depicts a perspective view of an illustrative valve body;

FIG. 6A depicts a top-down partial perspective view of an illustrative two-way valve;

FIG. 6B depicts a bottom-up partial perspective view of an illustrative two-way valve;

FIG. 7A depicts front and rear perspective views of an illustrative two-way valve in a first position;

FIG. 7B depicts front and rear perspective views of an illustrative two-way valve in a midpoint position;

FIG. 7C depicts front and rear perspective views of an illustrative two-way valve in a second position; and

FIG. 8 depicts an illustrative barb connector and outlet passage.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a” “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

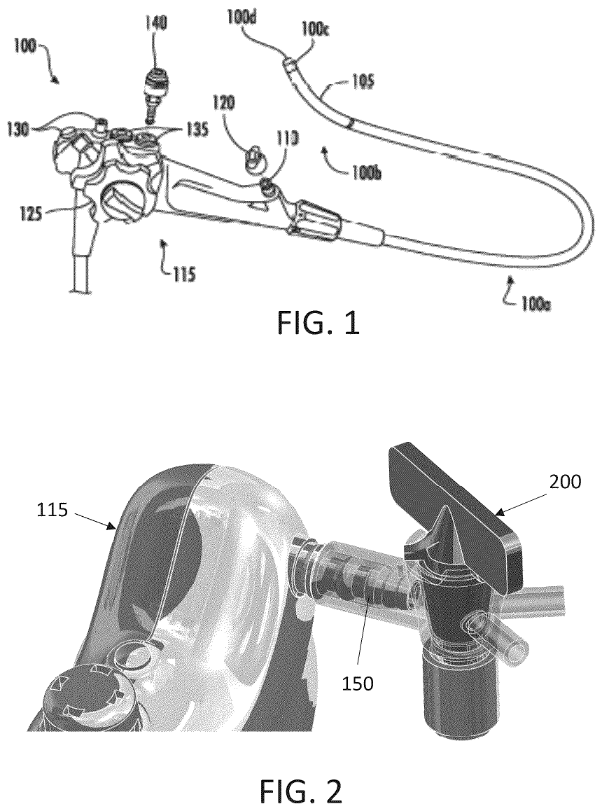

With reference to FIG. 1, an illustrative endoscope 100 is depicted. The endoscope 100 may include an elongated tube or shaft 100a that is configured to be inserted into a subject (e.g., a patient).

A light source may feed illumination light to a distal portion 100b of the endoscope 100. The distal portion 100b of the endoscope 100 may house an imager (e.g., CCD or CMOS imager) (not shown). The light source (e.g., lamp) may be located in a video processing unit that processes signals input from the imager and outputs processed video signals to a video monitor for viewing.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/suction nozzle for supplying gas to insufflate the interior of the patient at the treatment and to remove fluids via suction during treatment. An irrigation opening in the end face 100d supplies irrigation fluid to the treatment area of the patient. Illumination windows (not shown) that convey illumination light to the treatment area may also be included on the face 100d of the distal tip 100c. A working channel may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit (not shown) may be arranged on a proximal end side of the handle 115.

The handle 115 may be provided with dual valve locations 135. One of the valve locations 135 may receive a fluid valve 140 for operating an insufflating gas feed operation and suction line, as further described herein. The other valve location 135 may receive a water valve 145 connected to an irrigation line and source of irrigation fluid (not shown).

Within the operating handle 115, the fluid valve 140 may be in fluid communication with an input port for receiving a source of gas and/or suction. The input port may be in the form of a barb connector 150, as shown in FIG. 2. A two-way valve 200 includes an outlet passage (further described below) that is sized to receive the barb connector 150 of the operating handle 115. The position of the two-way valve 200 then determines whether the working line, including the barb 115, the fluid valve 135, and the gas/suction nozzle of the endoscope 100, acts as an insufflation line or as a suction line.

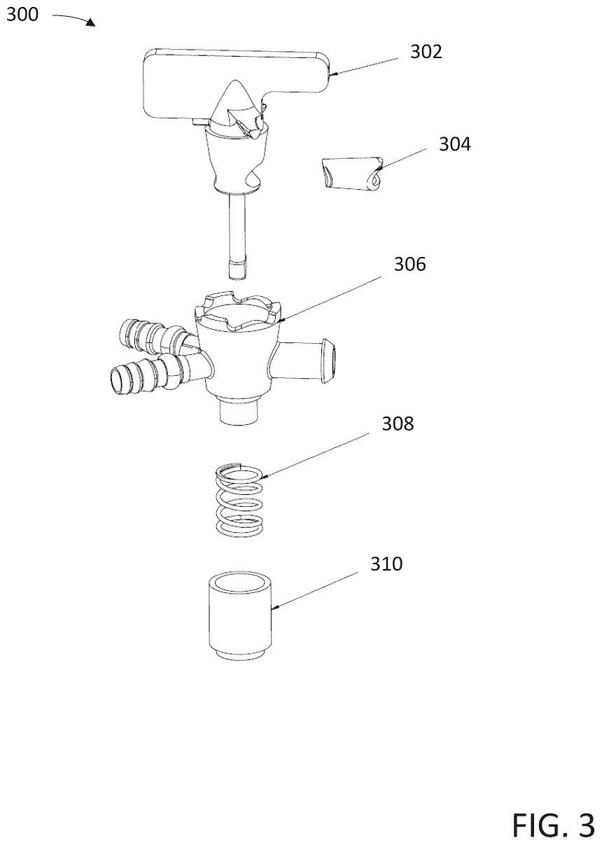

As shown in FIG. 3, a two-way valve 300 includes a knob 302, tube 304, body 306, spring 308, and end cap 310. The body 306 sits within the end cap 310, and the knob 302 is attached to and pulled towards the end cap 310 by means of the spring 308.

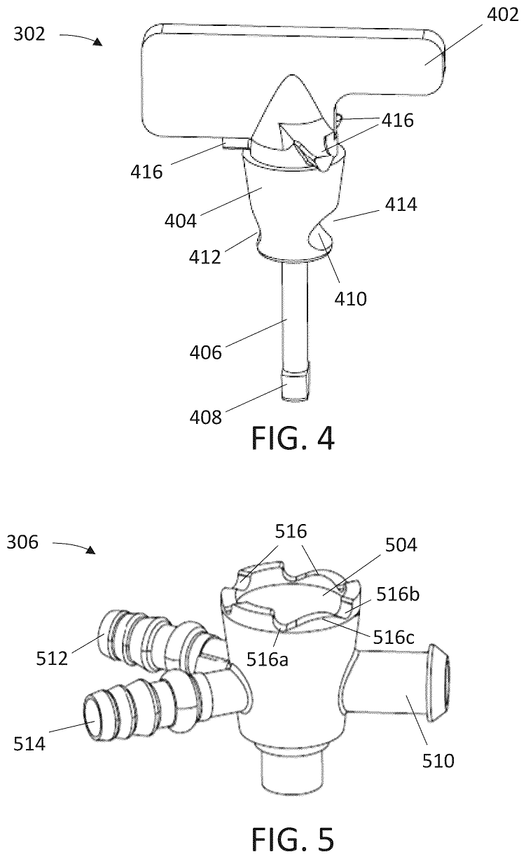

FIG. 4 shows the knob 302, which includes a knob handle 402 sized and positioned to be grasped and turned by one hand. Although shown the knob handle 402 is shown as a flat tab extending vertically, one of ordinary skill will understand that other shapes and positions are possible.

Below the handle 402 is the knob barrel 404, which has a curved side wall to allow the knob 302 to rotate during operation. A pole 406 with a rectangular key fitting 408 extends from the bottom of the knob barrel 404. Considering the outer surface of the knob barrel 404 as a tapered cylinder with a vertical axis of rotational symmetry, the pole 406 is positioned at the center of that vertical axis of symmetry to allow for rotation of the knob 302.

The knob barrel 404 has a channel 410 that extends through two opposite openings 412 and 414 in the side wall. The outlet opening 414 is significantly wider than the inlet opening 412 so that the outlet opening maintains fluid communication with the corresponding passage of the valve body 306 when the knob 302 is rotated. For the outlet opening 414 to contact the same passage of the valve body 306 in both positions of the knob 302, the outlet opening 414 subtends an arc of the side wall at least equal to the total angle of rotation of the knob 302 between valve positions.

The tube 304 sits within and is mated to the channel 410 while the valve 300 is in use. In some implementations, the tube 304 is made of a deformable material with raised portions around the openings 412 and 414 to hold the tube 304 within the channel 410 and help provide a tight seal.

Above the knob barrel 404 at the base of the knob handle 402, guide pins 416 extend from the knob 302. The guide pins 416 extend radially outward farther than the knob barrel 404 so that they are stopped by the top of the valve body 306 when the barrel is inserted into the body. Although the valve design as shown includes three guide pins 416, one of ordinary skill will recognize that more or fewer guide features are possible.

The valve body 306 includes first and second inlet passages 510, 512 and one outlet passage 514 as shown in FIG. 5. The three passages 510, 512, and 514 each open into the valve well 504, which is also open on the top and bottom. The inner surface of the valve well 504 is a tapered cylinder shaped similarly to the side wall of the knob barrel 404 so that the barrel fits within the well as the knob poll 406 extends out through the bottom of the valve body 306. Along the top of the valve body 306 are three guide grooves 516 sized and positioned to accept the guide pins 416 of the valve when the knob barrel 404 is inserted within the valve well 504. Each guide groove 516 is curved, having first and second groove seats 516a and 516b representing the lowest points of each groove, separated by a raised portion 516c. Although the valve design as shown includes three guide grooves 516, one of ordinary skill will recognize that the number of guide grooves 516 will match that of the guide pins 416 and that more or fewer guide grooves are possible.

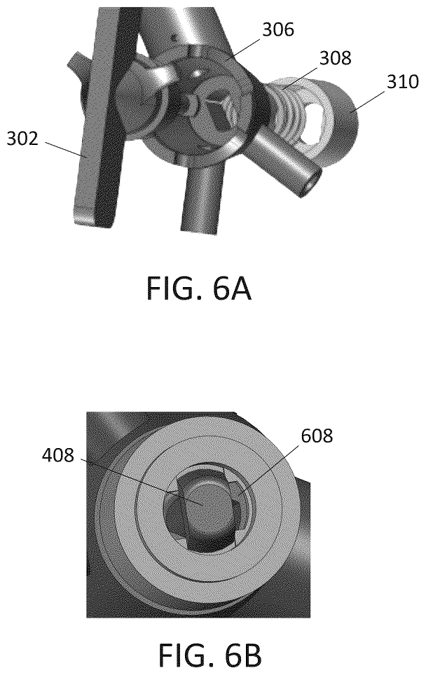

As shown in FIGS. 6A and 6B, the knob 302 is placed within the well of the valve body 306, at which point the key fitting 408 is inserted into the key opening 608 of the valve end cap 310 and then rotated to keep it in place. The spring 308 presses upward on the valve body 306 with respect to the knob 302 which is fixed in the cap 310, effectively pulling the knob 302 against the valve body 306.

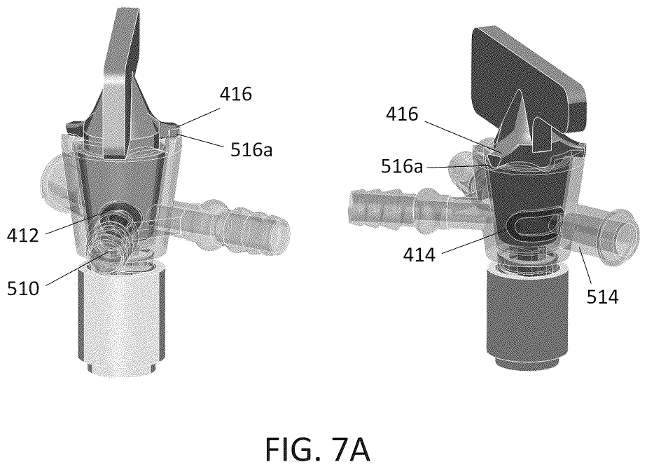

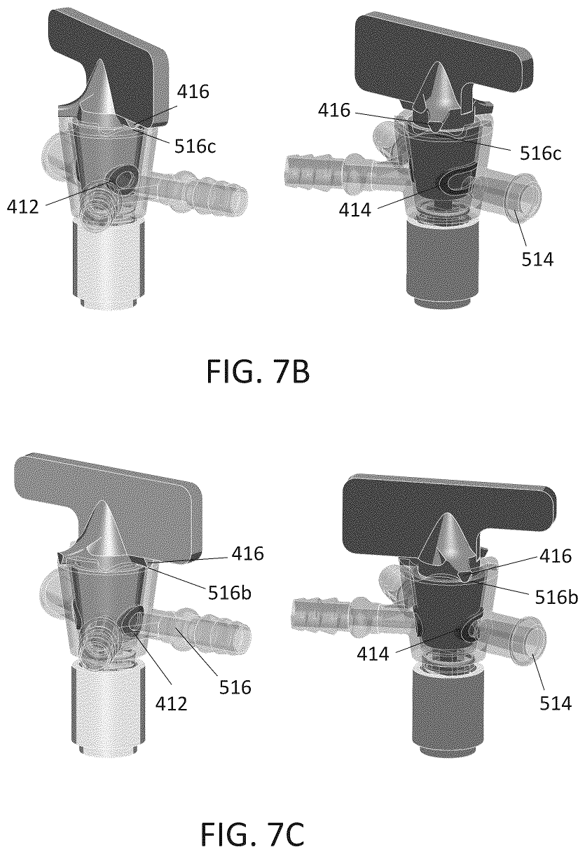

As the knob 302 is inserted into the well of the valve body 306, the guide pins 416 come in contact with the guide grooves 516. FIG. 7A shows the guide pins 416 in a first position in which the guide pins 416 are each positioned in the first seat 516a of their respective guide groove 516. The knob barrel 404 is securely seated in the valve well 504. The force of the spring 308 presses the side wall of the knob barrel 404 against the inner surface of the valve well 504, ensuring a tight seal between the raised portion of the tube 304 surrounding the inlet opening 412 and the first inlet passage 510. The raised portion of the tube 304 surrounding the outlet opening 414 provides a similar seal with the outlet passage 514.

The first inlet passage 510 may be connected to, for example, a source of suction. The source of suction may be, for example, a suction pump that is activated and maintained throughout the procedure. Other sources of negative pressure may be available based on the setting and equipment.

FIG. 7B shows a midpoint position during transition of the valve 300 between the first position illustrated in FIG. 7A and a second position illustrated in FIG. 7C. The operator pulls and turns the handle 402 to move the knob 302 away from the first position, with the upward force on the knob 302 loosening the seal between the inlet opening 412 and the first inlet passage 510. The pins 416 are each in contact with the raised portion 516c of their respective guide groove 516 so that the knob barrel 404 is not seated against the valve well 504 during the transition. The midpoint position shown in FIG. 7B is unstable; the action of the spring 308 on the knob 302 leads the valve to either the first or second positions in which the knob barrel 404 is seated and one of the two inlet passages 510 or 512 are connected.

Once the knob 302 is rotated past the midpoint position, it is pulled towards the second position shown in FIG. 7C by the action of the guide pins 416 in contact with the guide grooves 516 curved downward towards the second seats 516b. Here, the knob barrel 404 is again firmly seated and pressed against the valve well 504, creating a tight seal between the inlet opening 412 and the second inlet passage 512. The wider outlet opening 414 is still sealed with the outlet passage 514 as above.

The second inlet passage 512 may be connected to, for example, a gas feed line suitable for insufflation, such as room air, contained air, or oxygen. In some implementations, an air pump may be activated and maintained during the procedure. Cartridges, tanks, or other contained air sources may be used, and other sources of positive air pressure may be available based on the setting and equipment.

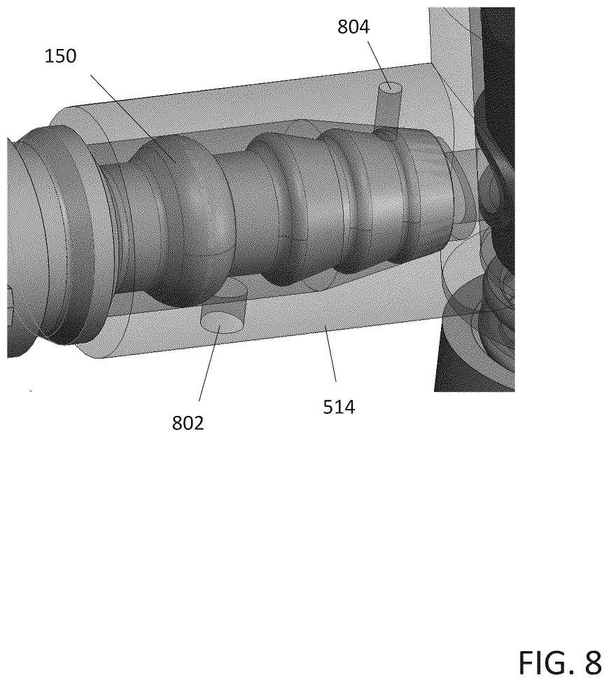

The endoscope handle 115 may connect to the two-way valve 300 in a variety of ways, such as by mating the barb connector 150 with the outlet passage 514. As shown in FIG. 8, the outlet passage 514 may include holes 802 and 804 from the outer surface to the inner surface of the passage 514. The holes 802 and 804 are radially opposite each other and axially offset. An adhesive is injected into the hole 802 and fills the spaces between the interior of the passage 514 and the exterior of the barb connector 150, coating multiple barbs to assure a solid adherence between the two components. As the space is filled, displaced air exits through the smaller hole 804.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A two-way valve assembly for a medical device, comprising:
a knob comprising:
a knob barrel having a side wall and an internal channel running between inlet and outlet openings in the side wall, and
one or more guide pins;
a valve body comprising:
a valve well encircling the knob barrel,
first and second inlet passages in fluid communication with the valve well,
an outlet passage in fluid communication with the valve well, and
one or more guide grooves, each guide groove receiving a different one of the one or more guide pins; and
a spring applying force to press the side wall of the knob barrel against an inner surface of the valve well;
wherein the knob rotates relative to the valve body between:
a first position in which the inlet opening in the knob barrel forms a seal with the first inlet passage in the valve body and the outlet opening in the knob barrel forms a seal with the outlet passage in the valve body, placing the first inlet passage in fluid communication with the outlet passage through the internal channel of the knob, and
a second position in which the inlet opening in the knob barrel forms a seal with the second inlet passage in the valve body and the outlet opening in the knob barrel forms a seal with the outlet passage in the valve body, placing the second inlet passage in fluid communication with the outlet passage through the internal channel of the knob; and
wherein each of the one or more guide pins moves from a first seat in a guide groove corresponding to the first position of the knob to a second seat of the guide groove corresponding to the second position of the knob when the knob rotates.

2. The valve assembly of claim 1, further comprising:
an end cap attached to the valve body opposite the knob, wherein the spring is attached to the valve body and the end cap to push the end cap away from the valve body, pressing the knob into the valve body.

3. The valve assembly of claim 2, wherein the knob further comprises a pole extending through the valve body and into the end cap, the pole having a key fitting at its distal end shaped to engage with a keyhole in the end cap.

4. The valve assembly of claim 1, further comprising a tube within the internal channel of the knob barrel, the tube having raised portions at the inlet and outlet openings to form a seal when the knob barrel is pressed against the valve well.

5. The valve assembly of claim 1, wherein the outlet opening of the knob barrel subtends an angle of the side wall equal to or greater than the angle of rotation between the first position and the second position.

6. The valve assembly of claim 1, wherein the guide grooves are each shaped with a raised portion between the first seat corresponding to the first position of the knob and the second seat corresponding to the second position of the knob such that, when rotating the knob between the first position and the second position, the knob barrel is lifted relative to the valve well to break contact between the knob barrel and the valve well.

7. The valve assembly of claim 1,
wherein the one or more guide pins is a plurality of guide pins extending radially from the knob and spaced with radial symmetry about the knob; and
wherein the one or more guide grooves is a plurality of guide grooves shaped and spaced with radial symmetry about the valve body.

8. The valve assembly of claim 1, wherein the side wall of the knob barrel and the inner wall of the valve body are cylinders tapered from top to bottom.

9. The valve assembly of claim 1, wherein the knob has a handle sized and shaped to be grasped and rotated by one hand.

10. A medical system for bronchoscopy, comprising:
an endoscope having a fluid channel therein;
a source of suction;
a gas source;
a two-way valve assembly comprising:
a knob comprising a knob barrel having a side wall and an internal channel running between inlet and outlet openings in the side wall and one or more guide pins;
a valve body comprising a valve well encircling the knob barrel, a first inlet passage in fluid communication with the source of suction and the valve well, a second inlet passage in fluid communication with the gas source and the valve well, an outlet passage in fluid communication with the endoscope fluid channel and the valve well, and one or more guide grooves, each guide groove receiving a different one of the one or more guide pins; and
a spring applying force to press the side wall of the knob barrel against an inner surface of the valve well;
wherein the knob rotates relative to the valve body between:
a first position in which the inlet opening in the knob barrel forms a seal with the first inlet passage in the valve body and the outlet opening in the knob barrel forms a seal with the outlet passage in the valve body, placing the source of suction in fluid communication with the endoscope fluid channel through the first inlet passage, the internal channel of the knob, and the outlet passage, and
a second position in which the inlet opening in the knob barrel forms a seal with the second inlet passage in the valve body and the outlet opening in the knob barrel forms a seal with the outlet passage in the valve body, placing the gas source in fluid communication with the endoscope fluid channel through the second inlet passage, the internal channel of the knob, and the outlet passage.

11. The medical system of claim 10, wherein each of the one or more guide pins moves from a first seat in a guide groove corresponding to the first position of the knob to a second seat of the guide groove corresponding to the second position of the knob when the knob rotates.

12. The medical system of claim 10, wherein the endoscope is a bronchoscope suitable for diagnostic and/or surgical deployment in the human respiratory system.

13. The medical system of claim 10, wherein the gas source is a source of gas suitable for insufflation during bronchoscopy.

14. The medical system of claim 10, wherein the gas is room air, contained air, or oxygen.

15. The medical system of claim 10, the valve assembly further comprising:
an end cap attached to the valve body opposite the knob, wherein the spring is attached to the valve body and the end cap to push the end cap away from the valve body, pressing the knob into the valve body.

16. The medical system of claim 10, wherein the knob of the valve assembly further comprises a pole extending through the valve body and into the end cap, the pole having a key fitting at its distal end shaped to engage with a keyhole in the end cap.

17. The medical system of claim 10, the valve assembly further comprising a tube within the internal channel of the knob barrel, the tube having raised portions at the inlet and outlet openings to form a seal when the knob barrel is pressed against the valve well.

18. The medical system of claim 10, wherein the outlet opening of the knob barrel subtends an angle of the side wall equal to or greater than the angle of rotation between the first position and the second position.

19. The medical system of claim 11, wherein the guide grooves are each shaped with a raised portion between the first seat corresponding to the first position of the knob and the second seat corresponding to the second position of the knob such that, when rotating the knob between the first position and the second position, the knob barrel is lifted relative to the valve well to break contact between the knob barrel and the valve well.

20. A method for operating a medical system, comprising:
deploying an endoscope into the respiratory system of a patient, the endoscope having a fluid channel therein connected to a two-way valve assembly, wherein the two-way valve assembly comprises:
a knob comprising a knob barrel having a side wall and an internal channel running between inlet and outlet openings in the side wall and one or more guide pins;
a valve body comprising a valve well encircling the knob barrel, a first inlet passage in fluid communication with a source of suction and the valve well, a second inlet passage in fluid communication with a source of air or oxygen and the valve well, an outlet passage in fluid communication with the endoscope fluid channel and the valve well, and one or more guide grooves, each guide groove receiving a different one of the one or more guide pins; and
a spring applying force to press the side wall of the knob barrel against an inner surface of the valve well;
while the knob of the two-way valve assembly is in a first position in which the inlet opening in the knob barrel forms a seal with the first inlet passage in the valve body and the outlet opening in the knob barrel forms a seal with the outlet passage in the valve body, clearing fluid from the respiratory system of the patient via suction through the endoscope fluid channel;
rotating the knob of the two-way valve assembly from the first position to a second position in which the inlet opening in the knob barrel forms a seal with the second inlet passage in the valve body and the outlet opening in the knob barrel forms a seal with the outlet passage in the valve body; and
insufflating the patient with air or oxygen deployed through the endoscope fluid channel.
